(19) United States
(12) Patent Application Publication
(10) Pub. No.: US 2014/0357939 A1
(43) Pub. Date: Dec. 4, 2014

(54) Meditation Device

(71) Applicant: Marc Frans Theeuwes, Felton, CA (US)

(72) Inventor: Marc Frans Theeuwes, Felton, CA (US)

(21) Appl. No.: 13/904,722

(22) Filed: May 29, 2013

(51) Int. Cl. A61M 21/02

(52) U.S. Cl. CPC A61M 21/02; A61M 2021/0027
USPC 600/27

(57) ABSTRACT

A meditation device including a body portion, electronics, and integrated user interface. The device includes a user interface that allows the user to set the meditation session duration, color of light, and choice of sound. The electronics enables the device to emit visual and auditory cues for when the meditation session begins and ends. The device is portable and easy to manufacture.

References Cited: U.S. PATENT DOCUMENTS

| PAT. NO. | Date | Inventor | Class |
|---|---|---|---|
| 4,755,908 | July, 1988 | Gardner | 361/326 |
| 4,776,323 | October, 1988 | Spector | 601/23 |
| 5,271,627 | December, 1993 | Russell | 273/445 |
| 5,722,418 | March, 1998 | Bro | 600/545 |
| 5,742,748 | April, 1998 | Sever | 345/419 |
| 5,792,025 | August, 1998 | Kikinis | 482/1 |
| 6,605,038 | August, 2003 | Teller | 600/300 |
| 6,846,270 | January, 2005 | Etnyre | 482/6 |
| 7,255,666 | August, 2007 | Cardenas | 482/143 |
| 7,297,093 | November, 2007 | Lopez | 482/124 |
| 7,485,071 | February, 2009 | Edwards | 482/23 |
| 7,488,282 | February, 2009 | Leavitt | 482/142 |
| 8,025,606 | September, 2011 | Hamilton | 482/4 |
| 8,096,923 | January, 2012 | White | 482/23 |
| 8,167,777 | May, 2012 | Nichols | 482/23 |
| 8,235,870 | August, 2012 | Hamilton | 482/4 |
| 8,298,126 | October, 2012 | Berc | 482/142 |

### FIELD OF THE INVENTION

This invention relates to apparatus for facilitating meditation practice. More specifically, the invention relates to a portable device that provides visual and or auditory queues for time based meditation.

### BACKGROUND OF THE INVENTION

Prior art have mentioned meditation benefits for many years. For example, Hamilton, U.S. Pat. No. 8,235,870 and related U.S. Pat. No. 8,025,606 contemplate a physical exercise apparatus comprising a mat/cushion device and accessories designed for fitness interactivity using an array of variations of graphics specifically placed on the mat where the physical exercise device, wherein said graphics are arranged to support proper positioning and guidance for multiple forms of physical exercise including meditation techniques. Hamilton U.S. Pat. No. 8,235,870 FIGS. 1A, 1B, and 5 of show a mat with icons that are used to guide an exercise routine. Leavitt, U.S. Pat. No. 7,488,282, contemplates an exercise device and method of use thereof, including an exercise platform and an incline base that can be used together or separately. That invention relates to a device for exercising, more particularly relating to a yoga prop used for multidimensional exercises such as yoga, strength-training, aerobics and meditation. Leavitt U.S. Pat. No. 7,488,282 FIGS. 1a, 1b, and 1c of said device illustrate a sizable apparatus for physical exercise including an exercise platform and an incline base that can be used together or separately. Edwards, U.S. Pat. No. 7,485,071, contemplates composite yoga mats and straps comprising a fabric sheet and loop holes and/or filament loops functioning as hand grips or appendage supports useful in the practice of yoga positions that functionally support practitioners in the learning and practice of yoga positions and meditation. Similarly U.S. Pat. Nos. 7,485,071, 7,297,093, 7,255,666, 6,846,270, help to align posture. These prior art mention meditation as a side benefit of using said inventions.

Teller, U.S. Pat. No. 6,605,038, provides a system for detecting, monitoring and reporting physiological information that includes a sensor device adapted to be worn on the upper arm that includes at least one of an accelerometer, a GSR sensor and a heat flux sensor and generates data indicative of at least one of activity, galvanic skin response and heat flow. Kikinis U.S. Pat. No. 5,792,025, contemplates a system to reduce repetitive motion injury risks, comprising a wrist-resting device having one or both of a heater and a vibrator in the device where such device can include software routines that launch a media player that presents on a computer video exercises, meditation, music, or a combination of any or all of these activities. Sever U.S. Pat. No. 5,742,748, also contemplates an invention that provides feedback through a computer program operable within a virtual reality device which is designed for perfecting mental visualization within the mind of a subject. Bro, U.S. Pat. No. 5,722,418, contemplates an invention for mediating social and behavioral influence processes through an interactive telecommunications guidance system. Russell, U.S. Pat. No. 5,271,627, contemplates a multidimensional equilateral angular game playing apparatus and method that includes electronically controlled, life-sized obelisk “opponents” in the vertex of each angle. And, Spector, U.S. Pat. No. 4,776,323, contemplates a biofeedback system to train an exerciser while who carries out athletic activity in which the arm or feet members move rhythmically and a system that translates this movement into an audible musical rhythm which makes it possible for the exerciser to be move in harmony with a musical rhythm conducive to optimal conditions of exercise.

U.S. Pat. No. 4,755,908, Gardner, Jul. 5, 1988 contemplates a capacitor comprising a sealed hollow glass container, inductor means, a portion of which is mounted within said sealed glass container, two or more layers of material deposited on the exposed surfaces contained within the sealed glass container and a gaseous mixture filling said sealed glass container having a pressure, at 25.degree. C., of approximately 100 torr or more, the gases in said mixture being selected from a group of gases consisting of helium, neon, argon, krypton and xenon, which when used in implementation can cause oscillating electric and magnetic fields that produce a system that has several resonant frequencies, and which has been found to be beneficial in creating an environment for meditation.

Prior art mentions meditation as a side benefit, but none are developed specifically to facilitate a meditation practice. More specifically none are developed to provide time based meditation with visual and auditory cues. In addition, many of these devices are large and not highly portable. Some prior art also use fragile glass containers that require high voltage and current, and complex systems that include integrated mechanical pumps, high pressure, and high vacuum systems, or entail large systems that require video, computer, and projections systems.

Accordingly, it is an object of the present new and unique invention to facilitate a meditation practice. It is further the object of this invention to be conveniently sized to fit easily in a practitioner's purse. It is further the object of this invention to provide visual and or auditory queues to aid the meditation practice over a selectable period of time. Additional objects and advantages of the invention will be set forth in part in the description that follows, and in part will be obvious from the description, or may be learned by practice of the invention.

### SUMMARY OF THE INVENTION

To achieve the foregoing objects and in accordance with the purpose of the invention as embodied and broadly described herein, the device of this invention is for guiding meditation including a body portion having a substantially planar lower surface and an ornamental exterior case. The interior of the invention includes electronic circuitry that provides auditory and or visual cues to facilitate meditation. The invention further contains user interface elements that allow the user to set the duration of the meditation session. Auditory cues are provided by recorded sound samples stored in the devices memory or loaded into the device by the user from time to time. Visual cues are provided by lighting elements such as but not limited to a liquid crystal display, bulb, or light emitting diode (LED). Preferably, the device allows the user to set the color of the visual queue and sound of the auditory queue. It is also preferred that the user can select the duration of the meditation session.

### BRIEF DESCRIPTION OF THE DRAWINGS

The accompanying drawings which are incorporated in and constitute a part of this specification, illustrate one embodiment of the invention, and, together with the description, serve to explain the principles of the invention.

### Of the Drawings

FIG. 1 is a view of the invention;

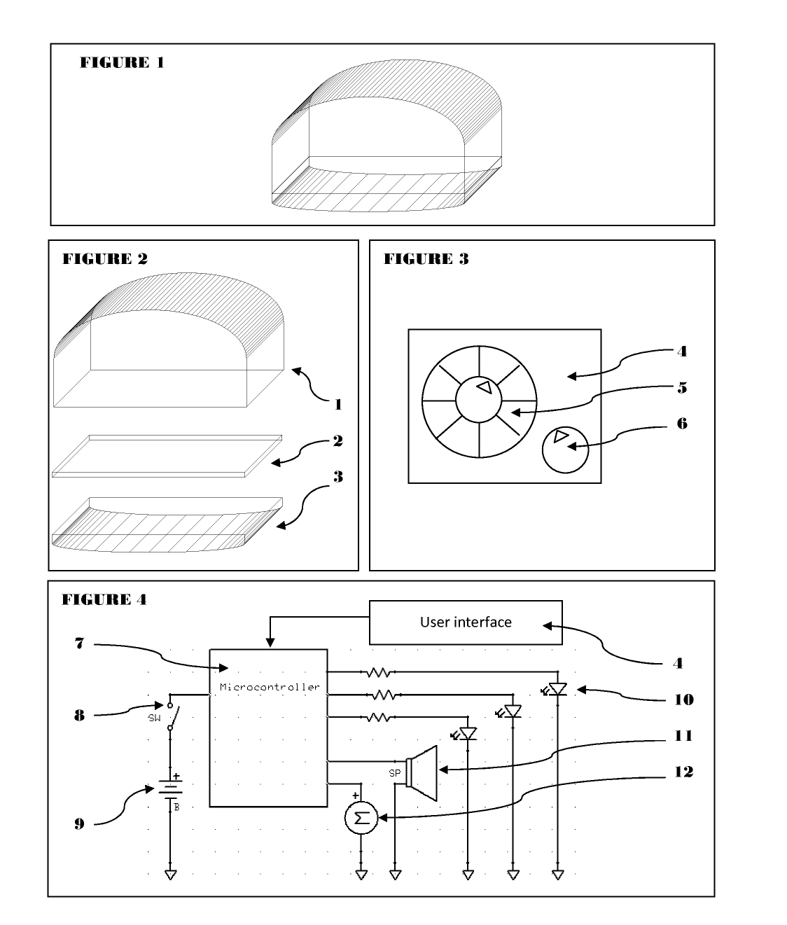

FIG. 2 is an exploded view of the invention;

FIG. 3 is a view of the user interface;

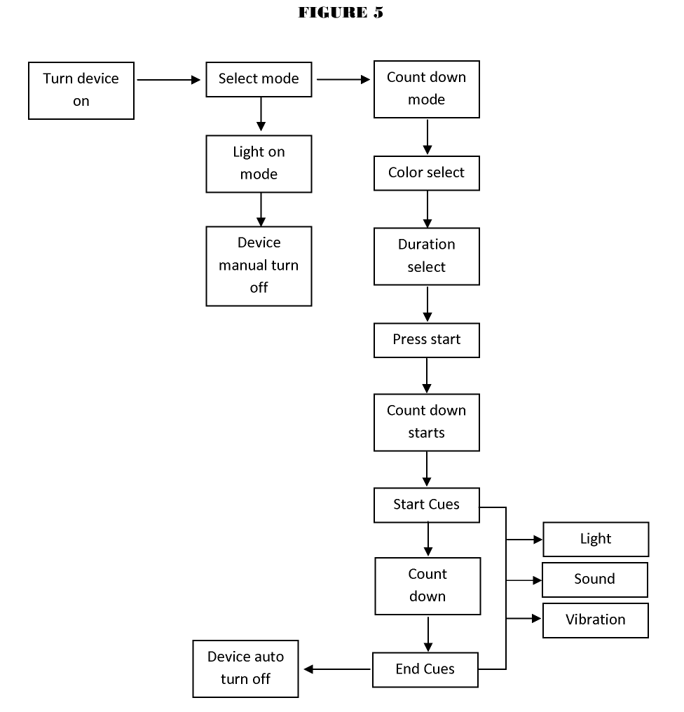

FIG. 4 is a schematic of the circuitry;

FIG. 5 is a schematic of the software logic;

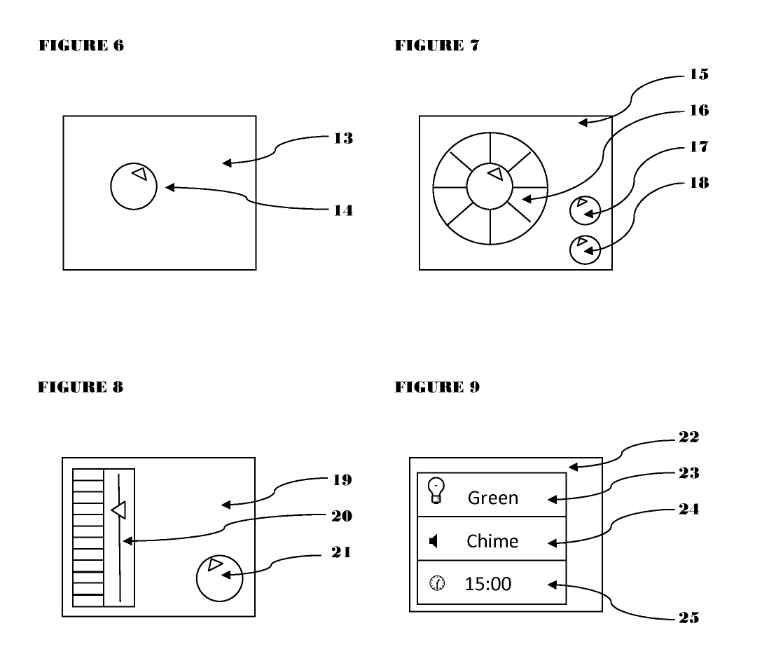

FIG. 6 is an alternate embodiment;

FIG. 7 is an alternate embodiment;

FIG. 8 is an alternate embodiment; and

FIG. 9 is an alternate embodiment.

### DESCRIPTION OF THE PREFERRED EMBODIMENT

Reference will now be made in detail to the present preferred embodiment of the invention, an example of which is illustrated in the accompanying drawings. As shown in FIG. 1, the invention is a meditation device. In accordance with the invention, the device contains a body, user interface, and electronic circuitry. As shown in FIG. 2, the body contains a top member 1 and bottom member 3 that enclose the circuit member 2. The top and bottom member can be connected via a snap fits, screws, or other fastening method that is common to those skilled in the art.

In accordance with the invention, the mediation device includes a user interface. As here embodied, the user interface includes general buttons that allow the user to set the meditation session duration and light color. As show in FIG. 3, the invention contains a user interface element 4 that allows the user to set the duration of the meditation session and choose the color for the light that emits during the meditation session.

A light selection wheel member 5 is turned to select the color of light to be emitted. Each section of member 5 contains a color. Color selection is made by turning a knob to the desired color. Those skilled in the art will appreciate the ability to use an alternative selection method such as but not limited to a capacitive touch panel or membrane switch panel commonly used in consumer electronics. A duration selection wheel member 6 is turned to set the duration of the meditation session.

In accordance with the invention, electronics are provided that count time and provide light, sound, and vibration. As embodied herein, the electronics includes a microcontroller, battery, switch, light emitting diodes, speaker, and motor. As show in FIG. 4, the invention contains electronic circuitry. A microcontroller member 7 contains the software that accepts input from the user interface element 4. A battery member 9 provides power and a switch member 8 turns the device on and off. A plurality or singular light emitting diode (LED) member 10 provides light during the meditation session. A single LED can be used to provide one color. A set of red, green, and blue LEDs can be used to create a myriad of colors. Those skilled in the art will appreciate the ability to create said myriad of colors by varying the voltage delivered by the microcontroller member 7 to the LED members 10. The voltage levels used with the set of red, green, and blue LED members 10 can be provided using pulse width modulation (PWM), which is common to those skilled in the art and described broadly in prior art. Sound is produced by the speaker member 11, and vibration is produced by a motor member 12.

In accordance with the invention the microcontroller member 7 contains software that operates as a routine as shown in FIG. 5. The software allows for a plurality of modes including but not limited to count down mode and light on mode. In this preferred embodiment if the countdown mode is selected, a color and duration is set. When start is pressed the software in microcontroller member 7 initiates the start cues that include light, sound, and vibration. Software then counts down until the end of the duration is reached. When the end of the duration is reached the software initiates end cues that include light, sound, and vibration and the device automatically turns off to conserve battery.

While the preferred embodiment described is particularly used with a described set of buttons, light and sound elements, the invention may expand or simplify the interface in other embodiments. For example, embodiment FIG. 6 provides a much simplified interface member 13 that only contains a duration selection wheel member 14. The embodiment in FIG. 7 provides an expanded interface member 15 that contains a light selection wheel member 16, duration selection wheel member 17, and sound selection wheel member 18.

It will be apparent to those skilled in the art that various modifications and variations can be made in the invention without departing from the scope or spirit of the invention. For example the embodiment in FIG. 8 provides an alternate interface member 19 that utilizes a light selection sliding switch member 20 in addition to the before mentioned duration selection wheel member 21, repeated in this figure for clarity. The embodiment in FIG. 9 provides an alternate interface member 22 that utilizes a liquid crystal display (LCD) that contains icons for setting the duration, sound, and color. For example, the LCD can use light section icon 23, sound section icon 24, and duration section icon 25. A touch sensing panel can be further integrated with the LCD allowing a user to select options from the LCD screen via finger press. It will be apparent to those skilled in the art that various modifications and variations can be made in the electronics without departing from the scope or spirit of the invention. For example a simplified circuit consisting of active and or passive components can be used to accomplish the device behavior noted in FIG. 5.

Thus, the invention provides a practical, novel, and useful device which may be economically manufactured, and which meets a need in the industry for meditation simplicity, utility, and portability.

What is claimed is:

1. A meditation device including a body portion, user interface, and electronics.

2. The user interface of claim 1 wherein said user interface allows the user to set the device time, color, and sound.

3. The electronics of claim 1 wherein said electronics includes electrical elements for monitoring time and providing light, sound, and vibration.

4. The electronics of claim 1 wherein said electronics contains a microcontroller that contains software that allows a user to operate the device in a plurality of modes.
